(19) United States
(12) Patent Application Publication
(10) Pub. No.: US 2025/0387634 A1
(43) Pub. Date: Dec. 25, 2025

(54) EXTENDABLE ACCESSORY CABLES

(71) Applicant: Stryker Corporation, Portage, MI (US)

(72) Inventors: Lillian Aranka Steiner, Kirkland, WA (US); Nasar Hussain, Kirkland, WA (US); Gretchen Nicole Rudel, Woodinville, WA (US)

(73) Assignee: Stryker Corporation, Portage, MI (US)

(21) Appl. No.: 19/244,747

(22) Filed: Jun. 20, 2025

Related U.S. Application Data
(60) Provisional application No. 63/662,827, filed on Jun. 21, 2024.

(51) Int. Cl. A61N 1/39; A61B 5/291

(52) U.S. Cl. CPC A61N 1/3968; A61B 5/291

(57) ABSTRACT

Systems, devices, and methods for using a retractable cable to connect an accessory device to a portable medical device are described herein. In some implementations, the retractable cable is retractably stored within a housing and is configured to connect the accessory device to the portable medical device. In some implementations, the retractable cable is configured to connect to a hub that is connected to the portable medical device. The example hub is, in various implementations, configured to connect to one or more retractable cable(s).

### CROSS-REFERENCE TO RELATED APPLICATION

This application claims priority to U.S. Provisional Patent Application No. 63/662,827 filed on Jun. 21, 2024, which is incorporated herein by reference in its entirety as if fully set forth herein.

### BACKGROUND

Portable medical devices are often connected to one or more accessory devices via wired connections. For example, a monitor-defibrillator may be connected, by a cable, to an accessory device configured to monitor or administer a treatment to a patient experiencing a medical emergency. The cable may be stored when not in use, such as in a bag, a case, or the like. In some examples, multiple cables may be stored together, for instance, in the same bag.

In various cases, the cables may be longer than necessary to ensure that the accessory device(s) can be routed and positioned on the patient in non-clinical environments and during patient transport. However, the cable length can result in the cables catching on other objects or becoming tangled with other cables. A cable may, in some instances, uncouple from a respective port, causing an interruption in monitoring and/or treatment of the patient. In some examples, an accessory device may malfunction while in use, and the accessory device may be replaced to continue monitoring and/or treatment of the patient.

### BRIEF DESCRIPTION OF THE DRAWINGS

FIGS. 1A and 1B illustrate example environments in which a portable medical device is connected to an accessory device that is disposed on a subject.

FIG. 2 illustrates an example retractable cable coupled to a portable medical device.

FIGS. 3A and 3B illustrate example processes for connecting accessory devices to a portable medical device.

FIG. 4 illustrates an example of an external defibrillator configured to perform various functions described herein.

### DETAILED DESCRIPTION

Implementations of the present disclosure are directed to specific improvements in the technical field of emergency medicine. In particular, implementations of the present disclosure relate to storing and connecting accessory devices to a portable medical device to monitor and/or treat a patient during a medical emergency.

Various implementations described herein relate to systems, devices, and methods for using a retractable cable to connect an accessory device to a portable medical device. In some implementations, the retractable cable is disposed within a housing and is configured to connect the accessory device to the portable medical device. In some implementations, the retractable cable is configured to connect to a hub that is connected to the portable medical device. The hub is, in various examples, configured to connect to one or more retractable cable(s). Various devices herein can provide an indication when a mechanical integrity or an electrical integrity of the retractable cable is degraded (e.g., due to use), and the retractable cable is ready to be replaced. Various devices described herein can enable the retractable cable to be extended and secured at a particular length. Accordingly, the retractable cable can be secured at a length for effectively positioning the accessory device on the patient. Various implementations described herein can enable organized storage of cables. The use of various implementations described herein can avoid delays in initiating use of accessory devices and improve patient care.

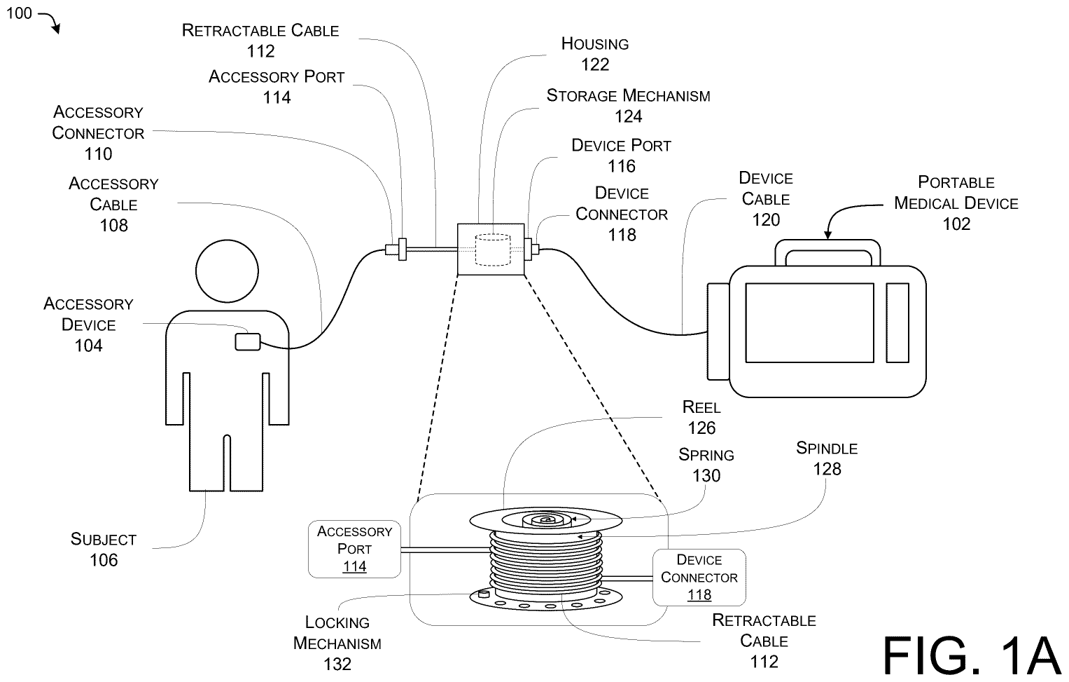

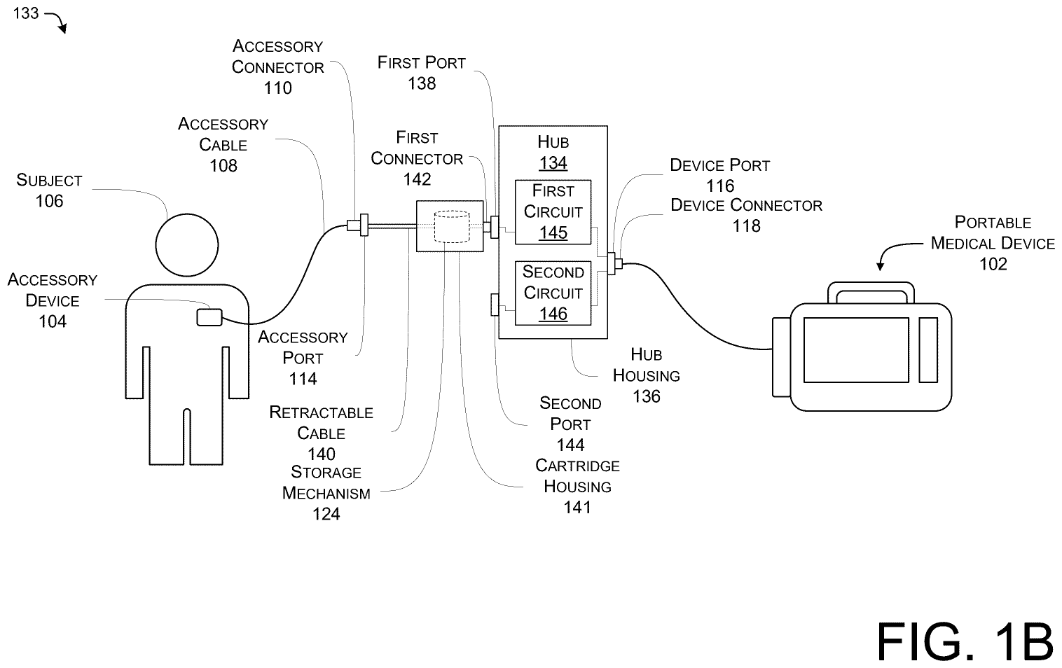

FIGS. 1A and 1B illustrate example environments in which a portable medical device 102 is connected to an accessory device 104 that is disposed on a subject 106. In various examples, the subject 106 is experiencing a medical emergency (e.g., cardiac arrest). The accessory device 104 is configured to, in various examples, detect a physiological parameter of the subject 106 and/or deliver a treatment to the subject 106.

Starting with FIG. 1A, the portable medical device 102 is configured to monitor and/or treat the subject 106. In some examples, the portable medical device 102 is configured to be operated outside of a clinical environment by a rescuer. For instance, the environment could be at the scene of a car crash, in an airport terminal, in a residence, or another non-clinical environment in which the subject 106 is experiencing the medical emergency. In some cases, a rescuer brings the portable medical device 102 to the subject 106 in response to a report that the subject 106 has lost consciousness. The rescuer is, in some examples, an untrained user, such as a bystander, who is present when the subject 106 experiences the medical emergency. In some examples, the rescuer is an emergency medical technician (EMT), a trained user, a nurse, or the like. The portable medical device 102, for instance, may monitor and/or treat the subject 106 in the environment before the subject 106 is transferred to a clinical care environment, such as a hospital, for further care. The portable medical device 102 may be a monitor-defibrillator, a defibrillator, an ECG monitor, a vital sign monitor, an ultrasound machine, or another type of portable medical device.

In various examples, the accessory device 104 is connected to the portable medical device 102. The accessory device 104, for instance, is disposed on the subject 106. In some cases, the accessory device 104 is configured to detect a physiological parameter of the subject 106 and/or administer a treatment to the subject 106. The accessory device 104 includes, in various examples, at least one of an electrode, a perfusion sensor, a blood oxygenation sensor, a blood flow sensor, a blood pressure sensor, an optical sensor, a capnography sensor, a motion sensor, a heart wall movement sensor, a sound sensor, an airway sensor, a pulse sensor, an ECG sensor, a temperature sensor, or any other appropriate sensor device. Examples of the physiological parameter, for instance, include an electrocardiogram (ECG), an impedance (e.g., a transthoracic impedance), a force administered to the patient, a blood pressure, an airway parameter (e.g., a capnograph, an end tidal gas parameter, a flow rate, etc.), a blood oxygenation (e.g., a pulse oximetry value, a regional oximetry value, a partial pressure of oxygen, etc.), a partial pressure of carbon dioxide, a capnograph, an electroencephalogram (EEG), a temperature, a heart sound, a blood flow rate, a physiological geometry (e.g., a shape of a blood vessel, an inner ear shape, etc.), a heart rate, a pulse rate, or another type of metric detected from the subject 106. In some cases, the accessory device 104 is configured to administer a treatment to the subject 106. For instance, the accessory device 104 may include one or more defibrillation electrodes, an airway adapter (e.g., connected to a ventilation device), a plunger or band for administering chest compressions to the subject 106, or some other device configured to apply a treatment to the subject 106.

In some instances, the accessory device 104 is electrically connected to an accessory cable 108. The accessory cable 108 is, in various cases, configured to provide, to the portable medical device 102, analog signals or data (e.g., digital signals) indicative of the analog signals detected by the accessory device 104. The accessory cable 108 may be any suitable cable known in the art. In some instances, the accessory cable 108 may include a conductive material (e.g., copper, aluminum, or the like) configured to transmit electrical signals and/or an insulative material (e.g., polytetrafluoroethylene (PTFE), polyurethane (PU), silicone, polyethylene (PE), polyvinyl chloride (PVC), ethylene vinyl acetate (EVA), thermoplastic elastomer (TPE), natural rubber, or the like) configured to shield the transmitted electrical signals from interference. The accessory cable 108, in some cases, may be a twisted pair cable, a coaxial cable, or another type of cable. In some instances, the accessory cable 108 may be selected based on durability, flexibility, shielding, conductivity, resistance, insulation, or any other characteristic that impacts performance. The accessory cable 108 is, in some examples, connected to an accessory connector 110. The accessory connector 110 may be configured to connect the accessory cable 108 to the portable medical device 102. Any cable described herein (including the accessory cable 108) may include at least one conductive wire that serves as at least one transmission interface. In some cases, a cable may include multiple conductive wires that respectively transmit different signals.

In various examples, it may be beneficial to be able to adjust the length of the accessory cable 108. For example, a rescuer may bring the portable medical device 102 and the accessory cable 108 to the subject 106 experiencing the medical emergency. The accessory cable 108, in some cases, is stored in a container (e.g., a bag, a case, or the like). In particular examples, more than one accessory cable 108 may be stored together in the container. The accessory cable(s) 108 may become tangled in storage, thereby causing a delay in initiating use of the accessory devices during the medical emergency.

In various examples, a length of the accessory cable 108 may be shorter or longer than a length between the portable medical device 102 and a position of the accessory device 104 on the subject 106. For instance, due to the length of the accessory cable 108, the accessory cable 108 may become caught on another object or become tangled with other cables that are being used for monitoring and/or treating the subject 106. In some instances, if a sensor is malfunctioning, it may be difficult to determine which sensor and/or cable corresponds to the malfunctioning sensor. If the cables are tangled, it may be difficult to identify and replace the malfunctioning sensor. In particular examples, the subject 106 and/or the portable medical device 102 may be moved during the medical emergency. The accessory cable 108 may catch on an object and, as a result, uncouple from the portable medical device 102. In some cases, the length of the accessory cable 108 may be insufficient to maintain the connection to the portable medical device 102 and the position of the accessory device 104 on the subject 106. Accordingly, the accessory cable 108 may uncouple from the portable medical device 102, thereby causing an interruption in the monitoring and/or treatment of the subject 106.

These issues can be addressed, in some cases, by using a retractable cable 112 that is configured to connect the accessory cable 108 to the portable medical device 102. The retractable cable 112 can be stored in a retracted configuration, thus avoiding the retractable cable 112 becoming tangled during storage. During the medical emergency, the rescuer can extend the retractable cable 112 from the retracted configuration and secure the retractable cable 112 at the necessary length to position the accessory device 104 on the subject 106. Accordingly, the use of the retractable cable 112 can avoid snagging, catching, and/or tangling of cables, thereby reducing disruptions in patient care. In some examples, the retractable cable 112 can be efficiently retracted after use to enable easier organization of cables during storage.

In some implementations, the retractable cable 112 is flexible. For instance, the retractable cable 112 may have a radius of curvature in a range of about 0.25 to 25 centimeters (cm). In some cases, the retractable cable 112 has a radius of curvature in a range of about 1 to 16 cm. In some implementations, the retractable cable 112 includes a material with a Young's modulus in a range of about 0.01 to 10 GPa. In various examples, the retractable cable 112 includes a material with a Young's modulus in a range of about 0.1 to 5 GPa. The retractable cable 112, in various examples, includes silicone, PVC, polypropylene (PP), PE, PU, nylon, TPE, thermoplastic rubber (TPR), thermoplastic amides (TPA), modified polyphenylene ether (mPPE), polytetrafluoroethylene (PTFE), fluorinatedethylenepropylene (FEP), perfluoroalkoxy alkanes (PFA), poly (ethene-co-tetrafluoroethene) (ETFE), polyvinylidene fluoride (PVDF), neoprene, polyethylene terephthalate (PETE), ethylene propylene diene monomer (EPDM), ethylene propylene (EP), EVA, natural rubber, or any other suitable material. In some instances, a core of the retractable cable includes a conductive material configured to transmit electrical signals and/or an insulative material configured to shield the transmitted electrical signals from interference.

In various examples, the retractable cable 112 is elastic. For instance, the retractable cable 112 may be configured to extend to a length (i.e., an extended length) that is longer than a resting length of the retractable cable 112. The resting length of the retractable cable 112 is, in various examples, the length of the retractable cable 112 when the retractable cable 112 is not stretched. Accordingly, the retractable cable 112 can be extended from the resting length and secured at the necessary length to position the accessory device 104 on the subject 106. When detached from the subject 106, the retractable cable 112 may spontaneously recoil to the resting length. The retractable cable 112 may include an elastic material. In some examples the retractable cable 112 includes an elastic material, such as rubber, elastane, latex, or the like. In some examples, the retractable cable 112 includes a material that is woven or braided, such as PP, polyester, nylon, PVC, vinyl, or the like. For instance, the woven material may be configured to extend from a resting length of the woven material.

The retractable cable 112, in some cases, is connected, by an accessory port 114, to the accessory connector 110 of the accessory cable 108. In various examples, the retractable cable 112 is connected, by a device port 116, to a device connector 118 of a device cable 120 that is connected to the portable medical device 102. In some examples, the retractable cable 112 is connected to the portable medical device 102. For instance, the portable medical device 102 may include a cable port, and the retractable wire may be connected to a cable connector configured to couple to the cable port. In some examples, the accessory connector 110, device connector 118, and the cable connector are the same type of connector. In some examples, the accessory port 114, the device port 116, and the cable port are the same type of port. The accessory connector 110, the device connector 118, and the cable connector are, in some cases, a custom connector, a USB connector, a serial connector, or another suitable connector. The device port 116 and the accessory port 114 are, in various instances, a custom port, a USB port, a serial port, or another suitable port. In some implementations, the retractable cable 112 is configured to connect to more than one accessory device 104. For instance, the retractable cable 112 may be connected to 1, 2, 3, 4, 5, or more than 5 accessory port(s) 114. The retractable cable 112, in some examples, may include multiple cables or wires within an outer sheath. The outer sheath may include an insulative material. In various cases, each cable or wire is connected to a separate accessory port. Each cable or wire may include a conductive material and an insulative material configured avoid interference between the transmitted electrical signals of each cable or wire. In some examples, the retractable cable 112 is connected to more than one accessory port 114. The retractable cable 112 may be configured to transmit electrical signals from each of the multiple accessory devices connected to the multiple accessory ports. In various examples, the retractable cable 112 may be configured to use multiplexing (e.g., time-division multiplexing, frequency-division multiplexing, code-division multiplexing, space-division multiplexing, or the like) or another technique configured to enable transmission of multiple signals. In some cases, the accessory port(s) 114 may be the same type of port. In some cases, the accessory port(s) 114 are different types of ports. The device port 116 or the cable connector, in various cases, is disposed within a housing 122.

In various implementations, the retractable cable 112 is disposed within the housing 122. The housing 122 may include any suitable material known in the art. In various examples, the housing 122 includes a rigid material. In some examples, the housing 122 may include a flexible material. For instance, the housing 122 may include a material that can bend at a radius of curvature in a range of about 1 to 55 cm. In some examples, the housing 122 includes a material that can bend at a radius of curvature in a range of about 2 to 25 cm. In some instances, the housing 122 includes a non-elastic material. For example, the housing 122 may include a material with a Young's modulus greater than about 0.1 GPa. In some cases, the housing 122 includes a material with a Young's modulus in a range of about 2 to 1500 GPa. In various examples, the housing 122 includes a material with a Shore hardness, as measured by a durometer, greater than 50 A. In some cases, the housing 122 includes a material with a Shore hardness in a range of 60 A to 100 A. The housing 122, in some examples, includes a material suitable for medical devices. For example, the housing 122 may include polycarbonate, acrylonitrile butadiene styrene, PP, PE, PETE, EVA, PU, TPE, natural rubber, neoprene, stainless steel, aluminum, carbon fiber, glass fiber, a combination thereof, or another suitable material.

In some examples, a first hole is disposed in the housing 122, and the retractable cable 112 is disposed through the first hole to enable the retractable cable 112 to connect to the accessory cable 108. In some examples, a second hole is disposed in the housing 122, and the retractable cable 112 is disposed through the second hole to enable the retractable cable 112 to connect to the portable medical device 102. The housing 122, in various instances, is connected to a storage mechanism 124.

The storage mechanism 124, in various implementations, is configured to retractably store the retractable cable 112. The storage mechanism 124 may include a reel 126, a spindle 128, a peg, or the like. For instance, the storage mechanism 124 may include the reel 126 that includes the spindle 128. When retracted, the retractable cable 112 may be disposed around a cylindrical surface of the spindle 128. The reel 126 and the spindle 128, for example, may include a rigid material. In some cases, the reel 126 and the spindle 128 include polycarbonate, acrylonitrile butadiene styrene, PP, PE, PETE, EVA, stainless steel, aluminum, carbon fiber, glass fiber, or another suitable material.

In some examples, a spring 130 is connected to the reel 126. For instance, an act of unwinding the retractable cable 112 from the spindle 128 rotates the reel 126, thereby at least partially unwinding the spring 130. When unwound, the spring 130 stores potential energy and is configured to spontaneously exert a torque on the reel 126. In various cases, the at least partially unwound spring 130 is configured to spontaneously retract the retractable cable 112 around the spindle 128 by rotating the reel 126.

In various implementations, the storage mechanism 124 is configured to store the retractable cable 112 in a z-fold configuration. For instance, the retractable cable 112 may be folded back and forth with the length of each segment between each of the folds being the same. In some examples, the length of each segment may be different. The retractable cable 112 may be secured in the z-fold configuration using, for example, a magnet, a clamp, a clip, a strap, a twist tie, a cable tie, a hook-and-loop fastener, or the like. In some implementations, the retractable cable 112 is secured in the z-fold configuration using multiple magnets and a spring-loaded mechanism. The multiple magnets may be disposed in a line, at a consistent interval between each of the multiple magnets. In some examples, each segment of the z-fold configuration includes a magnetic material configured to couple to one of the multiple magnets. The spring-loaded mechanism may be configured to expand and retract to secure the cable in the z-fold configuration. The retractable cable 112 may be extended from the z-fold configuration during the medical emergency. For instance, the storage mechanism 124 may include a first peg and a second peg. The retractable cable 112 may be disposed, in a repeated manner, around the first peg and the second peg. In some examples, the storage mechanism 124 may include a processor configured to extend and retract the retractable cable 112 from the first peg and the second peg. For instance, the processor may cause a notch on the first peg and/or the second peg to move, thereby causing the retractable cable 112 to extend from the storage mechanism 124. In some examples, the storage mechanism 124 may be configured to control the retraction and extension of the retractable cable 112. For example, the storage mechanism 124 may include a knob, a dial, a lever, a button, a switch, a slider, a joystick, a rotary encoder, a touchpad, a trackpad, or the like.

The storage mechanism 124, in some cases, includes a locking mechanism 132. The locking mechanism 132, in various examples, is configured to secure the retractable cable 112 at a particular retracted length. In some cases, the locking mechanism 132 enables the retractable cable 112 to be secured at particular intervals of extended lengths. For instance, the retractable cable 112 can be secured every 0.25, 0.5, 1, 2, 5, 7.5, 10, 12.5, or 15 cm. In various examples, the locking mechanism 132 enables the retractable cable 112 to be secured continuously. The locking mechanism 132, in some cases, includes a latch, a notch, a gear, a pin, a rachet and pawl mechanism, a cord lock, a clutch lock, a spring, or another suitable mechanism. In some cases, the locking mechanism 132 is configured to create friction on the retractable cable 112 to prevent retraction or extension of the retractable cable 112. The locking mechanism 132, in some cases, is configured to selectively prevent rotation of a component (e.g., the reel 126) of the storage mechanism 124. Accordingly, the accessory device 104 may be utilized while the accessory cable 108 is extended at a fixed length.

In some cases, the locking mechanism 132 includes a switch to retract the retractable cable 112 from an extended position. The switch, in various instances, releases the locking mechanism 132. For example, the switch may release a latch in the locking mechanism 132 holding the retractable cable 112 at a particular extended length. The switch may include a button, a toggle switch, a slide switch, a pin, or the like. In some cases, the switch is operated by the rescuer. For instance, the switch can be activated based on a button extending through the housing 122.

In various cases, the retractable cable 112 may degrade over time due to use. The retractable cable 112, in some examples, includes a sensor configured to determine when the retractable cable 112 is ready to be replaced. The sensor may detect an electrical characteristic, a mechanical characteristic, or a retraction of the retractable cable 112. For instance, the sensor may be configured to detect an electrical resistance, an electrical impedance, an electrical conductivity, a voltage reflection coefficient, or another electrical characteristic of the retractable cable 112. The sensor, in some cases, includes an ohmmeter, a Wheatstone bridge, a Maxwell bridge, a multimeter, an inductance, capacitance, and resistance (LCR) sensor, an impedance analyzer, a voltage sensor, a signal generator, a circuit, or the like. In some examples, the sensor may be configured to detect a mechanical resistance, a mechanical impedance, a thermal conductivity, or another mechanical characteristic of the retractable cable 112. The sensor, for example, may include a strain gauge, a piezoelectric element, a capacitive sensor, an optical sensor, an accelerometer, or the like.

In various implementations, the sensor is configured to detect a retraction of the retractable cable 112. The sensor may be a capacitive sensor, an inductive sensor, a magnetic sensor, an optical sensor, a tension sensor, a Hall effect sensor, a rotary encoder, or the like. In some examples, the sensor transmits a signal (e.g., data) indicative of the electrical characteristic, the mechanical characteristic, or a retraction of the retractable cable 112 to the processor or the portable medical device 102. In some cases, a sensor is configured to detect each time the retractable cable 112 is retracted. For instance, the sensor is coupled with the switch configured to cause retraction of the retractable cable 112. The sensor, in various implementations, is configured to provide a signal indicative of the switch being activated to the processor, to the portable medical device 102, or to some other external device. In some examples, the sensor is connected to a transceiver that is configured to transmit the signal to the processor, the portable medical device 102, or the external device. Accordingly, in various cases, the processor, the portable medical device 102, or the external device is configured to track the number of times the retractable cable 112 has been extended and/or retracted.

The processor and/or portable medical device 102, in various implementations, is configured to determine when the retractable cable 112 is ready for replacement. In some cases, the retractable cable 112 is ready for replacement when its electrical or mechanical characteristics have degraded. In some cases, a substantial number of retractions can permanently damage the retractable cable 112. When degraded, the retractable cable 112 may insert artifact into a signal transmitted by the retractable cable 112, such as a signal transmitted by the accessory device 104 to the portable medical device 102 (e.g., an artifact in data indicative of a detected physiological parameter) and/or a signal transmitted by the portable medical device 102 to the accessory device 104 (e.g., an artifact in an electrical shock output by the portable medical device 102). Accordingly, it may be preferred to replace the retractable cable 112 if the retractable cable 112 may interfere with monitoring and/or treatment of the subject 106. For instance, the processor may compare a use characteristic (e.g., the electrical characteristic, the mechanical characteristic, or the number of retractions) to a threshold value or a threshold range. The threshold value or the threshold range may be determined based on a known or use characteristic of the retractable cable 112. In some cases, if the use characteristic is greater than a first threshold or lower than a second threshold, the processor and/or the portable medical device 102 may determine that the retractable cable 112 is ready for replacement.

In various examples, the processor and/or the portable medical device 102 is configured to output an indication that the retractable cable 112 is ready for replacement. The processor or the portable medical device 102 may include a light emitter or an audio speaker configured to output a signal in response to the sensor detecting that the characteristic or the number of retractions is above or below the threshold value or outside the threshold range. For instance, the light emitter may turn on or begin flashing in response to the sensor determining that the number of retractions is above the threshold value.

In some implementations, a brush is configured to clean the retractable cable 112. In various cases, the brush may be attached to the housing 122 and/or to the storage mechanism 124. The brush, for instance, is configured to remove a contaminant (e.g., dust) from the retractable cable 112 when the retractable cable 112 is extended and/or retracted from the housing 122. The brush, in various instances, includes bristles, a fabric (e.g., polyester, nylon, rayon, or the like), a foam, a sponge, or the like. In some examples, the brush may include an antibacterial solution, antiseptic solution, or another solution configured to clean the retractable cable 112.

FIG. 1B illustrates an example of a hub 134 configured to connect one or more retractable cable(s) (e.g., the retractable cable 112) to the portable medical device 102. For example, the hub 134 may include the device port 116 configured to connect to the device connector 118 of the device cable 120 that is connected to the portable medical device 102. The hub 134, in some examples, includes a hub housing 136, and the device port 116 is disposed in the hub housing 136. In various cases, the hub 134 is connected to the portable medical device 102. The hub 134, for instance, may include a hub connector. The portable medical device 102, for instance, may include a hub port that is configured to couple to the hub connector. The hub connector is, in some examples, disposed in the hub housing 136.

In various implementations, the hub 134 includes a first port 138 configured to connect a first retractable cable 140 to the hub 134. The first retractable cable 140, in some cases, is disposed in a cartridge housing 141 (e.g., the housing 122). The storage mechanism 124 may be disposed within and/or attached to the cartridge housing 141.

The first port 138, in some examples, is configured to connect to a first connector 142 of the first retractable cable 140. The first port 138 may be disposed in the hub housing 136. The first retractable cable 140 is connected to, in various instances, the accessory port 114 that is configured to connect to the accessory connector 110 of the accessory cable 108. In some implementations, the hub 134 includes a second port 144 configured to connect a second retractable cable to the hub 134. In some examples, multiple retractable cables may be disposed within a cable housing (e.g., the housing 122). For instance, two retractable cables may be configured to connect to two accessory devices that are often used together. The two retractable cables may be disposed within one cable housing. In some examples, each of the multiple retractable cables are disposed within a separate cable housing. The device port 116, the first port 138, and the second port are, in various examples, a custom port, a USB port, a serial port, or the like. The device connector 118 and the first connector 142 are, in various examples, a custom connector, a USB connector, a serial connector, or the like.

A first circuit 145, in some cases, is connected between the device port 116 and the first port 138. The first circuit 145 is configured to transmit analog signals or data indicative of the analog signals from the accessory device 104 to the portable medical device 102. A second circuit 146, in some instances, is connected between the device port 116 and the second port 144. The first circuit 145 and the second circuit 146 are, in various instances, disposed within the hub housing 136. In some cases, the first circuit 145 and the second circuit 146 are electrically isolated from each other. For instance, the first circuit 145 and the second circuit 146 may be electrically isolated using a transformer, an optical isolator, a capacitor, or another technique known in the art. The first circuit 145 and the second circuit 146, in various cases, include an analog circuit and/or a digital circuit.

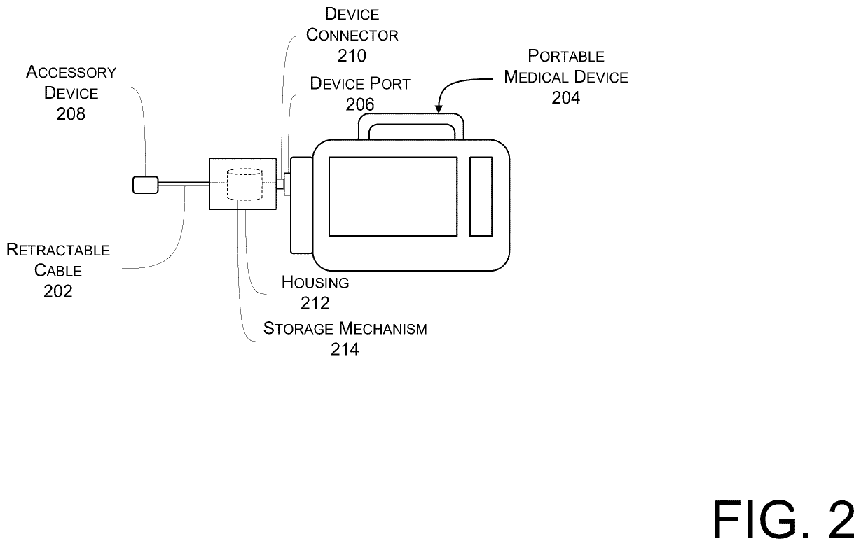

FIG. 2 illustrates an example retractable cable 202 coupled to a portable medical device 204. In various examples, the portable medical device 204 includes a device port 206. The retractable cable 202, in some cases, is connected between an accessory device 208 and a device connector 210 that is configured to couple to the device port 206. The retractable cable 202 may be disposed within a housing 212. In various implementations, the retractable cable 202 may be disposed within a housing of the portable medical device 204. In some examples, the housing 212 may be disposed within the housing of the portable medical device 204.

In some implementations, the device connector 210 is disposed in the housing 212. The device connector 210 may be a custom connector, a USB connector, a serial connector, or the like. The device port 206 may be a custom port, a USB port, a serial port, or the like. A hole may be disposed through the housing 212 to enable the retractable cable 202 to be extended by a storage mechanism 214.

In some examples, the retractable cable 202 is connected to a hub (e.g., the hub 134) that is configured to couple to the portable medical device 204. The hub may include the device connector 210. For instance, the device connector 210 may be disposed in a housing (e.g., the hub housing 136) of the hub. The retractable cable may include a connector (e.g., the first connector 142) that is configured to couple to a port (e.g., the first port 138) of the hub. The port is, in some examples, connected to the device connector 210 by a circuit (e.g., the first circuit 145). In various implementations, the hub is connected to more than one retractable cable. Each of the more than one retractable cables may be configured to connect to an accessory device or to an accessory cable that is connected to an accessory device.

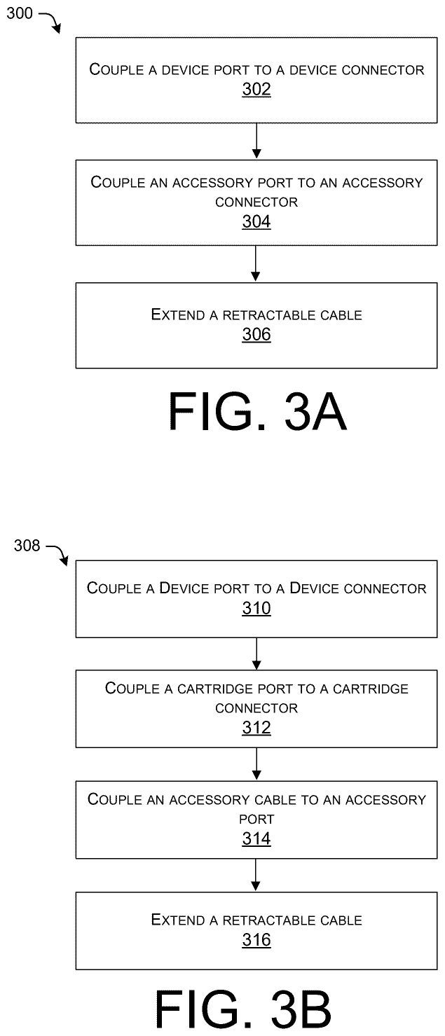

FIGS. 3A and 3B illustrate example processes 300 and 308 for connecting accessory devices to a portable medical device (e.g., the portable medical device 102). In some examples, the processes 300 and 308 can be performed by an entity, which may include a user (e.g., an untrained user, a bystander, an EMT, a physician, a nurse, or the like). According to some implementations, any of the steps of processes 300 and/or 308 may be omitted. In various implementations, the steps of processes 300 and/or 308 may be performed in a different order than illustrated in FIG. 3A and FIG. 3B, respectively.

Starting with FIG. 3A, at 302, a device port (e.g., the device port 116) connected to a retractable cable (e.g., a retractable cable 112) is coupled to a device connector (e.g., a device connector 118) connected to a device cable (e.g., the device cable 120). The retractable cable, in some examples, is disposed within a housing (e.g., the housing 122), and the device port is disposed in the housing. In some examples, the device port of the portable medical device is coupled to the device connector that is connected to the retractable cable.

At 304, an accessory port (e.g., an accessory port 114) connected to the retractable cable is coupled to an accessory connector (e.g., the accessory connector 110). The accessory connector, in various examples, is connected to an accessory cable (e.g., the accessory cable 108) that is connected to an accessory device (e.g., the accessory device 104) configured to monitor and/or administer a treatment to a subject (e.g., the subject 106) experiencing a medical emergency. The device port and the accessory port, in various examples, are a custom port, a USB port, a serial port, or the like. The device connector and the accessory connector, in some examples, are a custom connector, a USB connector, a serial connector, or the like. In some cases, the accessory port and the device port are the same type of port. The accessory port and the device port may be different types of ports. In various examples, the accessory connector and the device connector are the same type of connector. The accessory connector and the device connector may be different types of connectors. In some cases, the retractable cable may be connected to the accessory device, and step 304 may be omitted from the process 300.

At 306, the retractable cable extends by a storage mechanism (e.g., the storage mechanism 124). In various examples, the retractable cable may extend to position the accessory device on the subject. The retractable cable may be extended from a coiled configuration, a z-fold configuration, or the like. The retractable cable and the storage mechanism may be disposed in the housing, and the retractable cable may be disposed through a hole in the housing. The retractable cable may extend through the hole. In some examples, the retractable cable may extend, and a locking mechanism (e.g., the locking mechanism 132) may engage to secure the retractable cable at a particular retracted length.

In various implementations, the retractable cable may be retracted to the storage mechanism. The retractable cable may be retracted by a switch or by the locking mechanism being disengaged. In some cases, the retractable cable includes an elastic material and is configured to spontaneously recoil to a resting length when the accessory device is detached from the subject. The retractable cable may retract to a coiled configuration, a z-fold configuration, or the like. A brush may, in some examples, remove a contaminant when the retractable cable is retracted to or extended by the storage mechanism.

In some examples, a sensor detects that the retractable cable has degraded due to use and is ready to be replaced. For instance, the sensor may detect an electrical characteristic, a mechanical characteristic, or a retraction of the retractable cable 112. In some examples, the sensor provides the signal to a processor, the portable medical device, or an external device. The portable medical device, for instance, may output an alert, to the entity, that the retractable cable is ready to be replaced.

Moving to FIG. 3B, at 310, a device port of a hub (e.g., the hub 134) is coupled to a device connector connected to a device cable. In various examples, the device cable is coupled to a portable medical device. In some examples, the hub is coupled directly to the portable medical device. For instance, the portable medical device may include a device port configured to couple to a device connector of the hub. The hub, in various instances, includes a hub housing (e.g., the hub housing 136), and the device port or the device connector may be disposed in the hub housing.

At 312, a cartridge port (e.g., the first port 138) of the hub is coupled to a cartridge connector (e.g., the first connector 142) connected to a retractable cable. The cartridge port may be disposed in the hub housing. The retractable cable may be retractably stored in a storage mechanism, and the storage mechanism may be disposed within the cartridge housing. The cartridge connector, in some cases, is disposed in the cartridge housing.

At 314, an accessory cable is coupled to an accessory port connected to the retractable cable, in various implementations. The accessory port, in some examples, is connected to an accessory device configured to monitor and/or deliver a treatment to a subject experiencing a medical emergency. The device port, the cartridge port, and the accessory port may be a custom port, a USB port, a serial port, or the like. The device connector, the cartridge connector, and the accessory connector may be a custom connector, a USB connector, a serial connector, or the like. The device port, the cartridge port, and the accessory port, in various examples, are the same type of port. In some cases, the device port, the cartridge port, and the accessory port are different types of ports. The device connector, the cartridge connector, and the accessory connector, in various cases, are the same type of connector. In some instances, the device connector, the cartridge connector, and the accessory connector are different types of connectors.

At 316, the retractable cable extends by the storage mechanism. In some examples, a hole is disposed through the cartridge housing, and the retractable cable is disposed through the hole. The retractable cable may be extended through the hole. In various implementations, the retractable cable may extend, and a locking mechanism may engage to secure the retractable cable at a particular length.

In various implementations, a second cartridge port (e.g., the second port 144) of the hub is coupled to a second cartridge connector connected to a second retractable cable. A second accessory cable may couple to a second accessory port connected to the second retractable cable. The second accessory port, is come examples, is connected to a second accessory device. Accordingly, multiple accessory devices can be positioned on the subject.

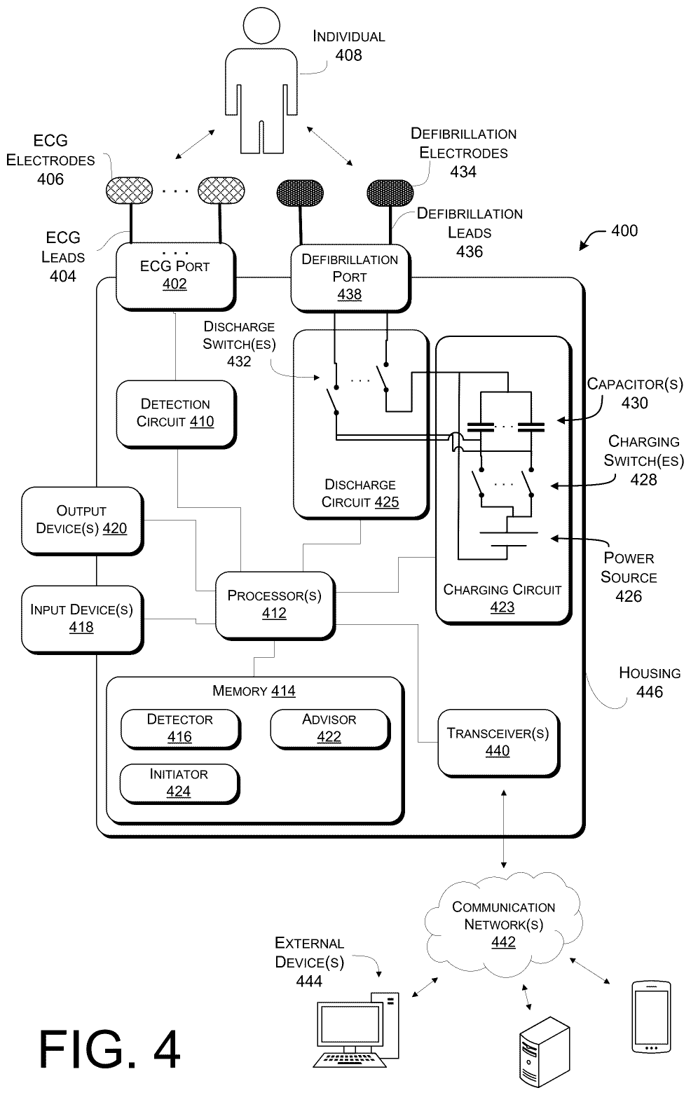

FIG. 4 illustrates an example of an external defibrillator 400 configured to perform various functions described herein. For example, the external defibrillator 400 is the portable medical device 102 described above with reference to FIGS. 1A and 1B.

The external defibrillator 400 includes an electrocardiogram (ECG) port 402 connected to multiple ECG leads 404. In some cases, the ECG leads 404 are removeable from the ECG port 402. For instance, the ECG leads 404 are plugged into the ECG port 402. The ECG leads 404 are connected to ECG electrodes 406, respectively. In various implementations, the ECG electrodes 406 are disposed on different locations on an individual 408. A detection circuit 410 is configured to detect relative voltages between the ECG electrodes 406. These voltages are indicative of the electrical activity of the heart of the individual 408.

In various implementations of the present disclosure, the multiple ECG leads 404 are connected to the external defibrillator 400 by a retractable cable (e.g., the retractable cable 112). In some implementations, the port 402 may be connected to a device connector (e.g., the device connector 118) of a device cable (e.g., the device cable 120). The device cable, in some examples, is configured to connect to the retractable cable. The multiple ECG leads 404 may be connected to one or more retractable cable(s). In some instances, the device cable is connected to a hub that is connected to one or more retractable cable(s). Each of the multiple ECG leads 404 may be connected to a respective retractable cable. In various examples, the retractable cable and/or the hub is connected to one or more additional accessory device(s) (e.g., a perfusion sensor, a blood oxygenation sensor, a blood flow sensor, a blood pressure sensor, an optical sensor, a capnography sensor, a motion sensor, a heart wall movement sensor, a sound sensor, an airway sensor, a pulse sensor, an ECG sensor, a temperature sensor, or the like).

In various implementations, the ECG electrodes 406 are in contact with the different locations on the skin of the individual 408. In some examples, a first one of the ECG electrodes 406 is placed on the skin between the heart and right arm of the individual 408, a second one of the ECG electrodes 406 is placed on the skin between the heart and left arm of the individual 408, and a third one of the ECG electrodes 406 is placed on the skin between the heart and a leg (either the left leg or the right leg) of the individual 408. In these examples, the detection circuit 410 is configured to measure the relative voltages between the first, second, and third ECG electrodes 406. Respective pairings of the ECG electrodes 406 are referred to as “leads,” and the voltages between the pairs of ECG electrodes 406 are known as “lead voltages.” In some examples, more than three ECG electrodes 406 are included, such that 5-lead or 12-lead ECG signals are detected by the detection circuit 410.

The detection circuit 410 includes at least one analog circuit, at least one digital circuit, or a combination thereof. The detection circuit 410 receives the analog electrical signals from the ECG electrodes 406, via the ECG port 402 and the ECG leads 404. In some cases, the detection circuit 410 includes one or more analog filters configured to filter noise and/or artifact from the electrical signals. The detection circuit 410 includes an analog-to-digital (ADC) in various examples. The detection circuit 410 generates a digital signal indicative of the analog electrical signals from the ECG electrodes 406. This digital signal can be referred to as an “ECG signal” or an “ECG.” The detection circuit 410, in some examples, generates a second digital signal indicative of the analog electrical signals from the one or more additional sensor(s). This second digital signal can be referred to as an “sensor signal.”

In some cases, the detection circuit 410 further detects an electrical impedance between at least one pair of the ECG electrodes 406. For example, the detection circuit 410 includes, or otherwise controls, a power source that applies a known voltage (or current) across a pair of the ECG electrodes 406 and detects a resultant current (or voltage) between the pair of the ECG electrodes 406. The impedance is generated based on the applied signal (voltage or current) and the resultant signal (current or voltage). In various cases, the impedance corresponds to respiration of the individual 408, chest compressions performed on the individual 408, and other physiological states of the individual 408. In various examples, the detection circuit 410 includes one or more analog filters configured to filter noise and/or artifact from the resultant signal. The detection circuit 410 generates a digital signal indicative of the impedance using an ADC. This digital signal can be referred to as an “impedance signal” or an “impedance.”

The detection circuit 410 provides the ECG signal and/or the impedance signal one or more processors 412 in the external defibrillator 400. In some implementations, the processor(s) 412 includes a central processing unit (CPU), a graphics processing unit (GPU), both CPU and GPU, or other processing unit or component known in the art.

The processor(s) 412 is operably connected to memory 414. In various implementations, the memory 414 is volatile (such as random access memory (RAM)), non-volatile (such as read only memory (ROM), flash memory, etc.) or some combination of the two. The memory 414 stores instructions that, when executed by the processor(s) 412, causes the processor(s) 412 to perform various operations. In various examples, the memory 414 stores methods, threads, processes, applications, objects, modules, any other sort of executable instruction, or a combination thereof. In some cases, the memory 414 stores files, databases, or a combination thereof. In some examples, the memory 414 includes, but is not limited to, RAM, ROM, electrically erasable programmable read-only memory (EEPROM), flash memory, or any other memory technology. In some examples, the memory 414 includes one or more of CD-ROMs, digital versatile discs (DVDs), content-addressable memory (CAM), or other optical storage, magnetic cassettes, magnetic tape, magnetic disk storage or other magnetic storage devices, or any other medium which can be used to store the desired information and which can be accessed by the processor(s) 412 and/or the external defibrillator 400. In some cases, the memory 414 at least temporarily stores at least one of the ECG signal, the impedance signal, or the sensor signal.

In various examples, the memory 414 includes a detector 416, which causes the processor(s) 412 to determine, based on the ECG signal and/or the impedance signal, whether the individual 408 is exhibiting a particular heart rhythm. For instance, the processor(s) 412 determines whether the individual 408 is experiencing a shockable rhythm that is treatable by defibrillation. Examples of shockable rhythms include ventricular fibrillation (VF) and ventricular tachycardia (V-Tach). In some examples, the processor(s) 412 determines whether any of a variety of different rhythms (e.g., asystole, sinus rhythm, atrial fibrillation (AF), etc.) are present in the ECG signal.

The processor(s) 412 is operably connected to one or more input devices 418 and one or more output devices 420. Collectively, the input device(s) 418 and the output device(s) 420 function as an interface between a user and the defibrillator 400. The input device(s) 418 is configured to receive an input from a user and includes at least one of a keypad, a cursor control, a touch-sensitive display, a voice input device (e.g., a microphone), a haptic feedback device (e.g., a gyroscope), or any combination thereof. The output device(s) 420 includes at least one of a display, a speaker, a haptic output device, a printer, or any combination thereof. In various examples, the processor(s) 412 causes a display among the input device(s) 418 to visually output a waveform of the ECG signal and/or the impedance signal. In some implementations, the input device(s) 418 includes one or more touch sensors, the output device(s) 420 includes a display screen, and the touch sensor(s) are integrated with the display screen. Thus, in some cases, the external defibrillator 400 includes a touchscreen configured to receive user input signal(s) and visually output physiological parameters, such as the ECG signal, the impedance signal, and the sensor signal.

In some examples, the memory 414 includes an advisor 422, which, when executed by the processor(s) 412, causes the processor(s) 412 to generate advice and/or control the output device(s) 420 to output the advice to a user (e.g., a rescuer). In some examples, the processor(s) 412 provides, or causes the output device(s) 420 to provide, an instruction to perform CPR on the individual 408. In some cases, the processor(s) 412 evaluates, based on the ECG signal, the impedance signal, or other physiological parameters, CPR being performed on the individual 408 and causes the output device(s) 420 to provide feedback about the CPR in the instruction. According to some examples, the processor(s) 412, upon identifying that a shockable rhythm is present in the ECG signal, causes the output device(s) 420 to output an instruction and/or recommendation to administer a defibrillation shock to the individual 408.

The memory 414 also includes an initiator 424 which, when executed by the processor(s) 412, causes the processor(s) 412 to control other elements of the external defibrillator 400 in order to administer a defibrillation shock to the individual 408. In some examples, the processor(s) 412 executing the initiator 424 selectively causes the administration of the defibrillation shock based on determining that the individual 408 is exhibiting the shockable rhythm and/or based on an input from a user (received, e.g., by the input device(s) 418. In some cases, the processor(s) 412 causes the defibrillation shock to be output at a particular time, which is determined by the processor(s) 412 based on the ECG signal and/or the impedance signal.

The processor(s) 412 is operably connected to a charging circuit 423 and a discharge circuit 425. In various implementations, the charging circuit 423 includes a power source 426, one or more charging switches 428, and one or more capacitors 430. The power source 426 includes, for instance, a battery. The processor(s) 412 initiates a defibrillation shock by causing the power source 426 to charge at least one capacitor among the capacitor(s) 430. For example, the processor(s) 412 activates at least one of the charging switch(es) 428 in the charging circuit 423 to complete a first circuit connecting the power source 426 and the capacitor to be charged. Then, the processor(s) 412 causes the discharge circuit 425 to discharge energy stored in the charged capacitor across a pair of defibrillation electrodes 434, which are in contact with the individual 408. For example, the processor(s) 412 deactivates the charging switch(es) 428 completing the first circuit between the capacitor(s) 430 and the power source 426, and activates one or more discharge switches 432 completing a second circuit connecting the charged capacitor 430 and at least a portion of the individual 408 disposed between defibrillation electrodes 434.

The energy is discharged from the defibrillation electrodes 434 in the form of a defibrillation shock. For example, the defibrillation electrodes 434 are connected to the skin of the individual 408 and located at positions on different sides of the heart of the individual 408, such that the defibrillation shock is applied across the heart of the individual 408. The defibrillation shock, in various examples, depolarizes a significant number of heart cells in a short amount of time. The defibrillation shock, for example, interrupts the propagation of the shockable rhythm (e.g., VF or V-Tach) through the heart. In some examples, the defibrillation shock is 200 J or greater with a duration of about 0.015 seconds. In some cases, the defibrillation shock has a multiphasic (e.g., biphasic) waveform. The discharge switch(es) 432 are controlled by the processor(s) 412, for example. In various implementations, the defibrillation electrodes 434 are connected to defibrillation leads 436. The defibrillation leads 436 are connected to a defibrillation port 438, in implementations. According to various examples, the defibrillation leads 436 are removable from the defibrillation port 438. For example, the defibrillation leads 436 are plugged into the defibrillation port 438.

In various implementations, the processor(s) 412 is operably connected to one or more transceivers 440 that transmit and/or receive data over one or more communication networks 442. For example, the transceiver(s) 440 includes a network interface card (NIC), a network adapter, a local area network (LAN) adapter, or a physical, virtual, or logical address to connect to the various external devices and/or systems. In various examples, the transceiver(s) 440 includes any sort of wireless transceivers capable of engaging in wireless communication (e.g., radio frequency (RF) communication). For example, the communication network(s) 442 includes one or more wireless networks that include a 3^{rd} Generation Partnership Project (3GPP) network, such as a Long Term Evolution (LTE) radio access network (RAN) (e.g., over one or more LTE bands), a New Radio (NR) RAN (e.g., over one or more NR bands), or a combination thereof. In some cases, the transceiver(s) 440 includes other wireless modems, such as a modem for engaging in WI-FI®, WIGIG®, WIMAX®, BLUETOOTH®, or infrared communication over the communication network(s) 442.

The defibrillator 400 is configured to transmit and/or receive data (e.g., ECG data, impedance data, sensor data, data indicative of one or more detected heart rhythms of the individual 408, data indicative of one or more defibrillation shocks administered to the individual 408, etc.) with one or more external devices 444 via the communication network(s) 442. The external devices 444 include, for instance, mobile devices (e.g., mobile phones, smart watches, etc.), Internet of Things (IoT) devices, medical devices, computers (e.g., laptop devices, servers, etc.), or any other type of computing device configured to communicate over the communication network(s) 442. In some examples, the external device(s) 444 is located remotely from the defibrillator 400, such as at a remote clinical environment (e.g., a hospital). According to various implementations, the processor(s) 412 causes the transceiver(s) 440 to transmit data to the external device(s) 444. In some cases, the transceiver(s) 440 receives data from the external device(s) 444 and the transceiver(s) 440 provide the received data to the processor(s) 412 for further analysis.

In various implementations, the external defibrillator 400 also includes a housing 446 that at least partially encloses other elements of the external defibrillator 400. For example, the housing 446 encloses the detection circuit 410, the processor(s) 412, the memory 414, the charging circuit 423, the transceiver(s) 440, or any combination thereof. In some cases, the input device(s) 418 and output device(s) 420 extend from an interior space at least partially surrounded by the housing 446 through a wall of the housing 446. In some instances, the housing 446 is configured to attach to a second housing (e.g., the housing 122, the hub housing 136). In various examples, the housing 446 acts as a barrier to moisture, electrical interference, and/or dust, thereby protecting various components in the external defibrillator 400 from damage.

In some implementations, the external defibrillator 400 is an automated external defibrillator (AED) operated by an untrained user (e.g., a bystander, layperson, etc.) and can be operated in an automatic mode. In automatic mode, the processor(s) 412 automatically identifies a rhythm in the ECG signal, makes a decision whether to administer a defibrillation shock, charges the capacitor(s) 430, discharges the capacitor(s) 430, or any combination thereof. In some cases, the processor(s) 412 controls the output device(s) 420 to output (e.g., display) a simplified user interface to the untrained user. For example, the processor(s) 412 refrains from causing the output device(s) 420 to display a waveform of the ECG signal and/or the impedance signal to the untrained user, in order to simplify operation of the external defibrillator 400.

In some examples, the external defibrillator 400 is a monitor-defibrillator utilized by a trained user (e.g., a clinician, an emergency responder, etc.) and can be operated in a manual mode or the automatic mode. When the external defibrillator 400 operates in manual mode, the processor(s) 412 cause the output device(s) 420 to display a variety of information that may be relevant to the trained user, such as waveforms indicating the ECG data and/or impedance data, sensor data (e.g., data from the one or more additional sensor(s), data from a sensor configured to detect an integrity of the retractable cable(s), data from a sensor configured to detect a retraction of the retractable cable(s)) notifications about detected heart rhythms, and the like.

### EXAMPLE CLAUSES

Various implementations of the present disclosure are described in the following Example Clauses.

1. A cartridge including: a housing; a monitor port disposed in the housing and configured to be physically coupled to a monitor connector of a device cable that is connected to a monitor-defibrillator; an electrocardiogram (ECG) port being configured to be physically coupled to an ECG connector of an ECG cable that is connected to an electrode; a connection cable connected between the monitor port and the ECG port; and a storage mechanism disposed in an interior space of the housing and configured to retractably store the connection cable.
2. The cartridge of clause 1, wherein the storage mechanism includes: a reel including a spindle, the connection cable being disposed around a cylindrical surface of the spindle; a spring physically connected to the reel and configured to rotate the reel; and a locking mechanism physically connected to the reel and configured to selectively prevent rotation of the reel.
3. The cartridge of clause 1 or 2, further including: a sensor configured to detect an electrical resistance of the connection cable; and a processor configured to: determine that the electrical resistance of the connection cable is outside a predetermined range; and output, to the monitor-defibrillator, an indication that the electrical resistance of the connection cable is outside the predetermined range.
4. A cartridge, including: a housing; a device port disposed in the housing and configured to be physically coupled to a device connector of a device cable that is connected to a portable medical device; a sensor port configured to be physically coupled to a sensor connector of a sensor cable that is connected to a sensor; a connection cable connected between the device port and the sensor port; and a storage mechanism disposed in an interior space of the housing and configured to retractably store the connection cable.
5. The cartridge of clause 4, wherein the connection cable includes silicone, polyvinyl chloride (PVC), polypropylene (PP), polyethylene (PE), polyurethane (PUR), nylon, thermoplastic elastomer (TPE), thermoplastic rubber (TPR), thermoplastic amides (TPA), modified polyphenylene ether (mPPE), polytetrafluoroethylene (PTFE), fluorinatedethylenepropylene (FEP), perfluoroalkoxy alkanes (PFA), poly (ethene-co-tetrafluoroethene) (ETFE), polyvinylidene fluoride (PVDF), neoprene, polyethylene terephthalate (PETE), ethylene propylene diene monomer (EPDM), or ethylene propylene (EP).
6. The cartridge of clause 4 or 5, wherein the sensor port is a first sensor port, wherein the sensor cable is a first sensor cable, and wherein the connection cable is connected to a second sensor port, the second sensor port being configured to physically couple to a second sensor connector of a second sensor cable.
7. The cartridge of any one of clauses 4-6, wherein the storage mechanism includes: a reel including a spindle, the connection cable being disposed around a cylindrical surface of the spindle; a spring physically connected to the reel and configured to rotate the reel; and a locking mechanism physically connected to the reel and configured to selectively prevent rotation of the reel.
8. The cartridge of clause 7, wherein the locking mechanism includes a latch, a notch, a gear, a pin, a ratchet and pawl mechanism, a cord lock, a clutch lock, or a spring.
9. The cartridge of any one of clauses 4-8, further including: a brush physically connected to the housing and configured to remove a contaminant from the connection cable.
10. The cartridge of any one of clauses 4-9, further including: a switch physically connected to the storage mechanism, the switch being configured to retract the connection cable into the storage mechanism.
11. The cartridge of any one of clauses 4-10, wherein the sensor is a first sensor, the cartridge further including: a second sensor configured to detect a retraction of the connection cable; and a processor configured to: determine a number of retractions of the connection cable; in response to determining the number of retractions, determine that the number of retractions is outside a predetermined range; and output an indication that the number of retractions is outside the predetermined range.
12. The cartridge of any one of clauses 4-11, wherein the sensor is a first sensor, the cartridge further including: a second sensor configured to detect an electrical resistance, an electrical impedance, or a voltage reflection coefficient of the connection cable; and a processor configured to: determine that the electrical resistance, the electrical impedance, or the voltage reflection coefficient is outside a predetermined range; and output an indication that the electrical resistance, the electrical impedance, or the voltage reflection coefficient is outside the predetermined range.
13. A method, including: coupling a device port to a device connector of a device cable that is connected to a portable medical device, the device port being disposed in a housing; coupling a sensor port to a sensor connector of a sensor cable that is connected to a sensor, the sensor port being connected, by a connection cable, to the device port; and extending, by a storage mechanism disposed in an interior space of the housing, the connection cable, the storage mechanism being configured to retractably store the connection cable.
14. The method of clause 13, wherein extending the connection cable includes: rotating a reel physically connected to the housing, the reel including a spindle; in response to rotating the reel, extending the connection cable, the connection cable being disposed around a cylindrical surface of the spindle; and stretching a spring physically connected to the reel; and engaging a locking mechanism physically connected to the reel and configured to prevent rotation of the reel.
15. The method of clause 14, wherein the locking mechanism includes a latch, a notch, a gear, a pin, a ratchet and pawl mechanism, a cord lock, a clutch lock, or a spring.
16. The method of any one of clauses 13-15, further including: retracting, by the storage mechanism, the connection cable into the storage mechanism.
17. The method of clause 16, further including: in response to retracting the connection cable, removing, by a brush physically connected to the housing, a contaminant on the connection cable.
18. The method of clause 16 or 17, wherein retracting the connection cable includes: disengaging a locking mechanism physically connected to a reel and configured to selectively prevent rotation of the reel, the reel being physically connected to the housing and including a spindle; in response to disengaging the locking mechanism, releasing a spring physically connected to the reel; in response to releasing the spring, rotating the reel; and in response to rotating the reel, wrapping the connection cable around a cylindrical surface of the spindle.
19. The method of any one of clauses 13-18, wherein the sensor is a first sensor, the method further including: detecting, by a second sensor, a retraction of the connection cable; in response to detecting a retraction, determining a number of retractions of the connection cable; in response to determining the number of retractions, determine that the number of retractions is outside a predetermined range; and outputting an indication that the number of retractions is outside the predetermined range.
20. The method of any one of clauses 13-19, wherein the sensor is a first sensor, the method further including: detecting, by a second sensor, an electrical resistance, an electrical impedance, or a voltage reflection coefficient of the connection cable; in response to detecting the electrical resistance, the electrical impedance, or a voltage reflection coefficient, determining that the electrical resistance or the electrical impedance is outside a predetermined range; and outputting an indication that the electrical resistance, the electrical impedance, or a voltage reflection coefficient is outside the predetermined range.
21. A hub, including: a hub housing; a monitor port disposed in the hub housing and configured to be physically coupled to a monitor connector of a monitor cable connected to a monitor-defibrillator; a cartridge port disposed in the hub housing; a circuit connected to the monitor port and the cartridge port; a cartridge including: a cartridge housing; a connection cable connected to an electrocardiogram (ECG) sensor port and configured to connect, via a connector, to the cartridge port; and a storage mechanism physically connected to the cartridge housing and configured to retractably store the connection cable; and an ECG sensor cable connected to an electrode and configured to be physically coupled, via an ECG sensor connector, to the ECG sensor port.
22. The hub of clause 21, wherein the storage mechanism includes: a reel including a spindle, the connection cable being disposed around a cylindrical surface of the spindle; a spring physically connected to the reel and configured to rotate the reel; and a locking mechanism physically connected to the reel and configured to prevent rotation of the reel.
23. The hub of clause 21 or 22, further including: a sensor configured to detect a retraction of the connection cable; and a processor configured to: determine a number of retractions of the connection cable; in response to determining the number of retractions, determine that the number of retractions is outside a predetermined range; and output, to the monitor-defibrillator, an indication that the number of retractions is outside the predetermined range.
24. A hub, including: a device port configured to be physically coupled to a device connector of a device cable that is connected to a portable medical device; a cartridge port; a circuit connected to the device port and the cartridge port; a cartridge including: a connection cable connected to an accessory port and configured to connect, via a cartridge connector, to the cartridge port, the accessory port being configured to physically couple to an accessory connector of an accessory cable that is connected to an accessory device; and a storage mechanism configured to retractably store the connection cable.
25. The hub of clause 24, wherein the connection cable includes silicone, polyvinyl chloride (PVC), polypropylene (PP), polyethylene (PE), polyurethane (PUR), nylon, thermoplastic elastomer (TPE), thermoplastic rubber (TPR), thermoplastic amides (TPA), modified polyphenylene ether (mPPE), polytetrafluoroethylene (PTFE), fluorinatedethylenepropylene (FEP), perfluoroalkoxy alkanes (PFA), poly (ethene-co-tetrafluoroethene) (ETFE), polyvinylidene fluoride (PVDF), neoprene, polyethylene terephthalate (PETE), ethylene propylene diene monomer (EPDM), or ethylene propylene (EP).
26. The hub of clause 24 or 25, wherein the accessory device includes an electrode, a perfusion sensor, a blood oxygenation sensor, a blood flow sensor, a blood pressure sensor, an optical sensor, a capnography sensor, a motion sensor, a heart wall movement sensor, a sound sensor, an airway sensor, a pulse sensor, an ECG sensor, or a temperature sensor.
27. The hub of any one of clauses 24-26, wherein the storage mechanism includes: a reel including a spindle, the connection cable being disposed around a cylindrical surface of the spindle; a spring physically connected to the reel and configured to rotate the reel; and a locking mechanism physically connected to the reel and configured to prevent rotation of the reel.
28. The hub of any one of clauses 24-27, further including a brush configured to remove a contaminant from the connection cable.
29. The hub of any one of clauses 24-28, further including: a sensor configured to detect a retraction of the connection cable; and a processor configured to: determine a number of retractions of the connection cable; in response to determining the number of retractions, determine that the number of retractions is outside a predetermined range; and output an indication that the number of retractions is outside the predetermined range.
30. The hub of any one of clauses 24-29, further including: a sensor configured to detect an electrical impedance, an electrical resistance, or a voltage reflection coefficient of the connection cable; and a processor configured to: determine that the electrical impedance, the electrical resistance, or a voltage reflection coefficient is outside a predetermined range; and output, to the portable medical device, an indication that the electrical impedance, the electrical resistance, or a voltage reflection coefficient is outside the predetermined range.
31. The hub of any one of clauses 24-30, further including: a switch physically connected to the storage mechanism and configured to retract the connection cable.
32. The hub of any one of clauses 24-31, wherein the cartridge port is a first cartridge port, the circuit is a first circuit, the cartridge is a first cartridge, the connection cable is a first connection cable, the accessory port is a first accessory port, the cartridge connector is a first cartridge connector, the accessory connector is a first accessory connector, the accessory cable is a first accessory cable, the accessory is a first accessory device, and the storage mechanism is a first storage mechanism, the hub further including: a second cartridge port; a second circuit connected to the device port and the second cartridge port; and a second cartridge including: a second connection cable connected to a second accessory port and configured to connect, via a second cartridge connector, to the second cartridge port, the second accessory port being configured to physically couple to a second accessory connector of a second accessory cable that is connected to a second accessory device; and a second storage mechanism configured to retractably store the second connection cable.
33. A method, including: coupling a device port to a device connector of a device cable that is connected to a portable medical device; coupling a cartridge port to a connector of a connection cable that is connected to an accessory port; and extending, by a storage mechanism, the connection cable, the storage mechanism being configured to retractably store the connection cable.
34. The method of clause 33, wherein the accessory port is configured to be physically coupled to an accessory connector of an accessory cable that is connected to an accessory device.
35. The method of clause 33 or 34, wherein extending the connection cable includes: rotating a reel, the reel including a spindle; in response to rotating the reel, extending the connection cable, the connection cable being disposed around a cylindrical surface of the spindle; and stretching a spring physically connected to the reel; and engaging a locking mechanism physically connected to the reel and configured to selectively prevent rotation of the reel.
36. The method of any one of clauses 33-35, further including: retracting, by the storage mechanism, the connection cable.
37. The method of clause 36, wherein retracting the connection cable includes: disengaging a locking mechanism physically connected to a reel and configured to selectively prevent rotation of the reel, the reel including a spindle; in response to disengaging the locking mechanism, releasing a spring physically connected to the reel; in response to releasing the spring, rotating the reel; and in response to rotating the reel, wrapping the connection cable around a cylindrical surface of the spindle.
38. The method of clause 36 or 37, further including: in response to retracting the connection cable, removing, by a brush, a contaminant from the connection cable.
39. The method of any one of clauses 33-38, further including: detecting, by a sensor, a retraction of the connection cable; in response to detecting a retraction of the connection cable, determining a number of retractions of the connection cable; in response to determining the number of retractions, determining that the number of retractions is outside a predetermined range; and outputting an indication that the number of retractions is outside the predetermined range.
40. The method of any one of clauses 33-39, further including: detecting, by a sensor, an electrical impedance, an electrical resistance, or a voltage reflection coefficient of the connection cable; in response to detecting the electrical impedance, the electrical resistance, or a voltage reflection coefficient, determining that the electrical impedance or the electrical resistance is outside a predetermined range; and outputting an indication that the electrical impedance, the electrical resistance, or a voltage reflection coefficient is outside the predetermined range.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain implementations are described herein, including the best mode known to the inventors for carrying out implementations of the disclosure. Of course, variations on these described implementations will become apparent to those of ordinary skill in the art upon reading the foregoing description. The inventors expect skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

1. A cartridge comprising:
a housing;
a monitor port disposed in the housing and configured to be physically coupled to a monitor connector of a device cable that is connected to a monitor-defibrillator;
an electrocardiogram (ECG) port being configured to be physically coupled to an ECG connector of an ECG cable that is connected to an electrode;
a connection cable connected between the monitor port and the ECG port; and
a storage mechanism disposed in an interior space of the housing and configured to retractably store the connection cable.

2. The cartridge of claim 1, wherein the storage mechanism comprises:
a reel comprising a spindle, the connection cable being disposed around a cylindrical surface of the spindle;
a spring physically connected to the reel and configured to rotate the reel; and
a locking mechanism physically connected to the reel and configured to selectively prevent rotation of the reel.

3. The cartridge of claim 1, further comprising:
a sensor configured to detect an electrical resistance of the connection cable; and
a processor configured to:
determine that the electrical resistance of the connection cable is outside a predetermined range; and
output, to the monitor-defibrillator, an indication that the electrical resistance of the connection cable is outside the predetermined range.

4. A cartridge, comprising:
a housing;
a device port disposed in the housing and configured to be physically coupled to a device connector of a device cable that is connected to a portable medical device;
a sensor port configured to be physically coupled to a sensor connector of a sensor cable that is connected to a sensor;
a connection cable connected between the device port and the sensor port; and
a storage mechanism disposed in an interior space of the housing and configured to retractably store the connection cable.

5. The cartridge of claim 4, wherein the connection cable comprises silicone, polyvinyl chloride (PVC), polypropylene (PP), polyethylene (PE), polyurethane (PUR), nylon, thermoplastic elastomer (TPE), thermoplastic rubber (TPR), thermoplastic amides (TPA), modified polyphenylene ether (mPPE), polytetrafluoroethylene (PTFE), fluorinatedethylenepropylene (FEP), perfluoroalkoxy alkanes (PFA), poly (ethene-co-tetrafluoroethene) (ETFE), polyvinylidene fluoride (PVDF), neoprene, polyethylene terephthalate (PETE), ethylene propylene diene monomer (EPDM), or ethylene propylene (EP).

6. The cartridge of claim 4, wherein the sensor port is a first sensor port,
wherein the sensor cable is a first sensor cable, and
wherein the connection cable is connected to a second sensor port, the second sensor port being configured to physically couple to a second sensor connector of a second sensor cable.

7. The cartridge of claim 4, wherein the storage mechanism comprises:
a reel comprising a spindle, the connection cable being disposed around a cylindrical surface of the spindle;
a spring physically connected to the reel and configured to rotate the reel; and
a locking mechanism physically connected to the reel and configured to selectively prevent rotation of the reel.

8. The cartridge of claim 7, wherein the locking mechanism comprises a latch, a notch, a gear, a pin, a ratchet and pawl mechanism, a cord lock, a clutch lock, or a spring.

9. The cartridge of claim 4, further comprising:
a brush physically connected to the housing and configured to remove a contaminant from the connection cable.

10. The cartridge of claim 4, further comprising:
a switch physically connected to the storage mechanism, the switch being configured to retract the connection cable into the storage mechanism.

11. The cartridge of claim 4, wherein the sensor is a first sensor, the cartridge further comprising:
a second sensor configured to detect a retraction of the connection cable; and
a processor configured to:
determine a number of retractions of the connection cable;
in response to determining the number of retractions, determine that the number of retractions is outside a predetermined range; and
output an indication that the number of retractions is outside the predetermined range.

12. The cartridge of claim 4, wherein the sensor is a first sensor, the cartridge further comprising:
a second sensor configured to detect an electrical resistance, an electrical impedance, or a voltage reflection coefficient of the connection cable; and
a processor configured to:
determine that the electrical resistance, the electrical impedance, or the voltage reflection coefficient is outside a predetermined range; and
output an indication that the electrical resistance, the electrical impedance, or the voltage reflection coefficient is outside the predetermined range.

13. A method, comprising:
coupling a device port to a device connector of a device cable that is connected to a portable medical device, the device port being disposed in a housing;
coupling a sensor port to a sensor connector of a sensor cable that is connected to a sensor, the sensor port being connected, by a connection cable, to the device port; and
extending, by a storage mechanism disposed in an interior space of the housing, the connection cable, the storage mechanism being configured to retractably store the connection cable.

14. The method of claim 13, wherein extending the connection cable comprises:
rotating a reel physically connected to the housing, the reel comprising a spindle;
in response to rotating the reel,
extending the connection cable, the connection cable being disposed around a cylindrical surface of the spindle; and
stretching a spring physically connected to the reel; and
engaging a locking mechanism physically connected to the reel and configured to prevent rotation of the reel.

15. The method of claim 14, wherein the locking mechanism comprises a latch, a notch, a gear, a pin, a ratchet and pawl mechanism, a cord lock, a clutch lock, or a spring.

16. The method of claim 13, further comprising:
retracting, by the storage mechanism, the connection cable into the storage mechanism.

17. The method of claim 16, further comprising:
in response to retracting the connection cable, removing, by a brush physically connected to the housing, a contaminant on the connection cable.

18. The method of claim 16, wherein retracting the connection cable comprises:
disengaging a locking mechanism physically connected to a reel and configured to selectively prevent rotation of the reel, the reel being physically connected to the housing and comprising a spindle;
in response to disengaging the locking mechanism, releasing a spring physically connected to the reel;
in response to releasing the spring, rotating the reel; and
in response to rotating the reel, wrapping the connection cable around a cylindrical surface of the spindle.

19. The method of claim 13, wherein the sensor is a first sensor, the method further comprising:
detecting, by a second sensor, a retraction of the connection cable;
in response to detecting a retraction, determining a number of retractions of the connection cable;
in response to determining the number of retractions, determine that the number of retractions is outside a predetermined range; and
outputting an indication that the number of retractions is outside the predetermined range.

20. The method of claim 13, wherein the sensor is a first sensor, the method further comprising:
detecting, by a second sensor, an electrical resistance, an electrical impedance, or a voltage reflection coefficient of the connection cable;
in response to detecting the electrical resistance, the electrical impedance, or a voltage reflection coefficient, determining that the electrical resistance or the electrical impedance is outside a predetermined range; and
outputting an indication that the electrical resistance, the electrical impedance, or a voltage reflection coefficient is outside the predetermined range.
